(19) United States
(12) Patent Application Publication
(10) Pub. No.: US 2025/0027071 A1
(43) Pub. Date: Jan. 23, 2025

(54) NOVEL LIPOCALIN-TYPE PROSTAGLANDIN D SYNTHASE (L-PGDS) MUTANT AND USES THEREOF

(71) Applicants: Nanyang Technological University, Singapore (SG); Singapore Health Services Pte Ltd, Singapore (SG)

(72) Inventors: Konstantin Pervushin, Singapore (SG); Jia Yi Kimberly Low, Singapore (SG); Jodhbir Singh Mehta, Singapore (SG); Bhargy Sharma, Singapore (SG)

(21) Appl. No.: 18/778,525

(22) Filed: Jul. 19, 2024

(30) Foreign Application Priority Data
Jul. 20, 2023 (SG) SG10202302056Y

(51) Int. Cl. C12N 9/90; A61K 38/00

(52) U.S. Cl. CPC C12N 9/90; C12Y 503/99002; A61K 38/00

(57) ABSTRACT

The present disclosure relates to the pharmaceutical field. Specifically, the present disclosure relates to a novel Lipocalin-type prostaglandin D synthase (L-PGDS) mutant (RA L-PGDS) as well as its use in the treatment of amyloid-related diseases.

### CROSS-REFERENCE TO RELATED APPLICATION

This application claims priority from Singapore Patent Application No. 10202302056Y filed on Jul. 20, 2023, the contents of which are incorporated herein in their entirety by reference.

### FIELD OF THE INVENTION

The present disclosure relates to the pharmaceutical field. Specifically, the present disclosure relates to a novel Lipocalin-type prostaglandin D synthase (L-PGDS) mutant (RA L-PGDS) as well as its use in the treatment of amyloid-related diseases.

### BACKGROUND OF THE INVENTION

The formation of amyloid deposits in different types of tissues is implicated in many degenerative disorders, such as Alzheimer's disease (AD) and corneal dystrophy (CD). Transforming growth factor beta induced (TGFBI)-related CD is the most common heritable stromal CD worldwide (Chao-Shern et al., 2019). It is characterized by the accumulation of insoluble protein deposits in the corneal tissues, eventually leading to the progressive loss of corneal transparency and blindness in patients (Lakshminarayanan et al., 2014). To date, there are more than 74 different mutations reported in the TGFBI gene, and the genotypes are linked to the different isoforms of the amyloid deposits observed in patients (Nielsen et al., 2020; Han et al., 2016; Kheir et al., 2019). Mutations in the TGFBI gene result in the mutant protein product, TGFBI protein (TGFBIp) which is proteolytically cleaved in the corneal tissues (Stone et al., 1994; Munier et al., 1997; Kannabiran et al., 2005). The cleaved peptides from the TGFBIp are primarily responsible for the formation of amyloid deposits as they are enriched in the amyloid corneal sample and highly prone to aggregation (Venkatraman et al., 2017; Anandalakshmi et al., 2017). Polymorphic amyloid degeneration is another form of the amyloid deposit-related corneal disorder that is non-inheritable. This non-inheritable trait of the disease shows that environmental and other factors besides genetics can be associated with corneal amyloid deposition (Mannis et al., 1981; Molia et al., 1999). This phenomenon is also observed in Alzheimer's disease (AD), a genetically complex disorder. It is associated with well-defined risk loci, notably the APOE*4 allele (Yu et al., 2014), as well as various environmental factors such as air pollution and bacterial infection (Kim et al., 2009; Underwood et al., 2017; Stephen et al., 2019). This evidence indicates that the occurrence of corneal amyloids might be more widespread than the current estimated number based on population genotyping, warranting urgent attention for both prevention and treatment strategies against corneal amyloid-related diseases.

The current treatment strategies available for TGFBI-related CD include invasive techniques such as surgical ablation of the diseased cornea or corneal transplantation. However, recurrence is common among patients even after the surgery. These shortcomings of current treatment approaches highlight the importance of the search for a long-term treatment against this disease (Han et al., 2016). Most of the amyloid deposits in these corneal diseases are found in the central area of the cornea where the blood supply is limited (Lee et al., 2006). Therefore, it is suggested that molecular factors such as protein chaperones found in the blood vessels might play a role in preventing the accumulation of amyloid deposits in the corneal tissues (Han et al., 2016). Tears and aqueous humour primarily nourish the corneal tissue and they contain several native chaperones such as HtrA1 and clusterin (Poulsen et al., 2019; Wilson et al., 2021). However, the protecting effect might not be sufficient to clear amyloid deposits in an adverse genetic or environmental background (Han et al., 2016). Conversely, the cerebrospinal fluid (CSF) has evolved to have a unique set of chaperones to deal with polymorphic amyloid deposits such as Aβ amyloids (Peinado et al., 2022). Since most amyloid deposits are found in the extracellular region where the amount of Adenosine triphosphate (ATP) is low, the majority of the amyloid chaperones in the CSF are ATP-independent (Kannaian et al., 2019; Meyer-Luehmann et al., 2013). Different types of chaperone machinery are evolved to restrict the formation and disaggregate fibrils. The two main types of chaperones consist of the ATP-dependent disaggregases such as the heat shock protein (Hsp) complexes and the passive disaggregases such as lipocalin-type prostaglandin D synthase (L-PGDS), HtrA1 and clusterin (Low et al., 2022). The chaperone Hsp104 complex utilizes energy from ATP hydrolysis to actively channel its amyloid substrates through the central pore of its complex. At the same time, Hsp70 forms a multimolecular complex on the amyloid resulting in its disassembly via entropic pull. The ATP energy is used to disassociate the complex from products of disaggregation and recycle the chaperone (Gates et al., 2017; De Los Rios et al., 2006). However, the mechanism of ATP-independent disaggregation remains unresolved.

L-PGDS is an ATP-independent disaggregase which exerts both inhibitory and amyloid disaggregation effects by directly binding to the monomeric Aβ peptides and preformed amyloid fibrils (Kannaian et al., 2019; Kanekiyo et al., 2007). L-PGDS is the second most abundant protein in the CSF and one of the critical chaperones present in the CSF but is absent in the corneal tissues (Kannaian et al., 2019; Reiber et al., 2001; Dyrlund et al., 2012). The absence of L-PGDS native expression in the corneal tissues suggests that its direct administration may result in toxicity and unwanted side effects.

Thus, there is still a need for a novel treatment of amyloid-related diseases such as AD and CD, which has disaggregation and/or fibril inhibition activities.

### SUMMARY OF THE INVENTION

The present invention relates to a novel L-PGDS mutant, RA L-PGDS, which shows significant effects in disaggregating TGFBIp fibrils and Aβ fibrils, inhibiting the formation of TGFBIp fibrils, and disaggregating bacterial amyloids, while maintaining the heme binding and peroxidase activity. The mutant polypeptide comprises the substitutions K58R+C65A of the wild-type L-PGDS polypeptide sequence. The C65 residue is crucial for the catalytic function and peroxidase activity of L-PGDS. Consequently, the absence of C65 in RA L-PGDS reduces its toxicity by minimizing unwanted interactions and activities within cells. Further, mutation of the lysine to arginine at position 58 in RA L-PGDS enhances its activity compared to wild-type or single mutant C65A L-PGDS, as the more positively charged arginine improves the interaction with fibrils. Thus, the advantageous properties of RA L-PGDS enable it to be used in the treatment of various amyloid-related diseases and in the inhibition of the growth of a fibril-producing bacteria.

Thus, in a first aspect of the invention, there is provided an isolated Lipocalin-type prostaglandin D synthase (L-PGDS) polypeptide mutant comprising the substitutions K58R+C65A of the wild-type L-PGDS polypeptide sequence and consisting of the amino acid sequence as set forth in SEQ ID NO: 5.

In a second aspect of the invention, there is provided a composition comprising the isolated L-PGDS polypeptide mutant of the first aspect and at least one pharmaceutically acceptable excipient, diluent or carrier.

In a third aspect of the invention, there is provided an in vitro method of inhibiting the growth of a fibril-producing bacteria, comprising contacting an effective amount of the L-PGDS polypeptide mutant of the first aspect or the composition of the second aspect of the present invention with the bacteria.

In some embodiments of the third aspect, the fibril-producing bacteria are present in medical devices, environment settings (such as soil, oil spill), food products, beverage products and personal care products.

In some embodiments of the third aspect, the fibril-producing bacteria is selected from a group consisting of Proteobacteria (Alpha-, Beta-, Gamma- and Delta-proteobacteria), Bacteriodetes, Chloroflexi, Firmicutes and Actinobacteria. In some embodiments, the fibril-producing bacteria is selected from a group consisting of Bacteroides fragilis, Chloroflexus aggregans, Bacillus licheniformis, Salmonella typhimurium, Enterobacter sakazaki, Aeromonas caviae, Xanthomonas anxidopidos, Chromobacterium violaceum, Burkholderia gladioli, Burkholderia pseudomallei, Ralstonia pikettii, Stenotrophomonas maltophilia, Escherichia coli (E. coli), Salmonella enterica, Pseudomonas aeruginosa, Pseudomonas fluorescens, Pseudomonas putida, Staphylococcus aureus and Mycobacterium tuberculosis.

In a fourth aspect of the invention, there is provided a method of treating amyloid-related diseases in a subject, comprising administering a therapeutically effective amount of the L-PGDS polypeptide mutant of the first aspect or the composition of the second aspect of the present invention to the subject.

In some embodiments of the fourth aspect, the amyloid-related disease is selected from corneal dystrophy (CD) such as TGFBI-related CD, Alzheimer's disease (AD), frontotemporal dementia (FTD), Down's syndrome (DS), Pick disease (PiD), argyrophilic grain disease (AGD), progressive supranuclear palsy (PSP), corticobasal degeneration (CBD), Huntington's disease (HD), Prion disease (PrD), cataracts, bacterial infections caused by fibril-producing bacteria, Type 2 diabetes and Parkinson's disease (PD).

In some embodiments of the fourth aspect, the CD is TGFBI-related CD. In some embodiments, the fibril-producing bacteria is selected from a group consisting of Proteobacteria (Alpha-, Beta-, Gamma- and Delta-proteobacteria), Bacteriodetes, Chloroflexi, Firmicutes and Actinobacteria. In some embodiments, the fibril-producing bacteria is selected from a group consisting of Bacteroides fragilis, Chloroflexus aggregans, Bacillus licheniformis, Salmonella typhimurium, Enterobacter sakazaki, Aeromonas caviae, Xanthomonas anxidopidos, Chromobacterium violaceum, Burkholderia gladioli, Burkholderia pseudomallei, Ralstonia pikettii, Stenotrophomonas maltophilia, Escherichia coli (E. coli), Salmonella enterica, Pseudomonas aeruginosa, Pseudomonas fluorescens, Pseudomonas putida, Staphylococcus aureus and Mycobacterium tuberculosis.

### BRIEF DESCRIPTION OF THE DRAWINGS

Certain embodiments of the present disclosure are described more fully hereinafter with reference to the accompanying drawings.

FIG. 1 shows the G623R disaggregation effects of L-PGDS derivatives. A. Relative ThT fluorescence intensity of 50 μM mature G623R fibrils without or with 10 μM WT, C65A or RA L-PGDS disaggregation treatments for 48 h. Data plotted are averages with SD (n=3). Statistical analysis performed with one-tailed Student's t-test, *p<0.05. B. Transmission electron micrographs of 50 μM mature G623R fibrils without or with 10 μM WT, C65A or RA L-PGDS disaggregation treatment for 48 h. Scale bar is 100 nm. C. Histogram of lengths of control G623R fibrils without or with 10 μM WT-L-PGDS or RA L-PGDS disaggregation treatments, measured from TEM images (n=15). D. Histogram of lengths of G623R fibrils with 10 μM WT-L-PGDS or C65A-L-PGDS disaggregation treatments, measured from TEM images (n=8).

FIG. 2 shows the G623R inhibitory effects of L-PGDS derivatives. A. Relative ThT fluorescence intensity of 50 μM monomeric G623R peptides without or with 10 μM WT, C65A or RA L-PGDS inhibitory treatment for 48 h. Data plotted are averages with SD (n=3). Statistical analysis performed with one-tailed Student's t-test, *p<0.05. B. Transmission electron micrographs of 50 μM monomeric G623R peptides without or with 10 μM WT, C65A or RA-L-PGDS disaggregation treatment for 48 h. Scale bar is 100 nm.

FIG. 3 shows Congo red intensity of the bacterial amyloid after incubation with RA L-PGDS. Absorbance of the amyloid-binding Congo Red dye detected at 488 nm wavelength in DH5a cell culture grown in nutrient agar before (left) and after treatment with 50 μM RA L-PGDS (right).

FIG. 4 shows the Aβ (1-40) disaggregation and inhibitory effects of L-PGDS mutants. A. Relative ThT fluorescence intensity of 50 μM mature Aβ (1-40) fibrils without or with 10 μM WT, C65A or RA L-PGDS disaggregation treatment for 24 h. Data plotted are averages with SD (n=3). Statistical analysis performed with one-tailed Student's t-test, *p<0.05. B. Relative ThT fluorescence intensity of 50 μM monomeric Aβ (1-40) peptides without or with 10 μM WT, C65A or RA L-PGDS inhibitory treatment for 24 h. Data plotted are averages with SD (n=3). Statistical analysis performed with one-tailed Student's t-test, *p<0.05.

FIG. 5 shows the heme binding and peroxidase activities of L-PGDS mutants. A. UV/Vis absorption spectra of 5 μM heme only or 5 M heme-L-PGDS derivative complexes. B. Peroxidase activity of 5 μM LPGDS derivative-heme complexes by observation of TMB oxidization at Abs652 nm. Activity was monitored for 1 h.

FIG. 6 shows the inhibition of TGFBIp peptide variants by WT L-PGDS and RA L-PGDS. A. Relative TGFBIp aggregation measured through ThT fluorescence of 50 μM four different TGFBIp mutant fibrils (515-533), (591-614), (571-588) and (R124C), in the presence of 5 μM WT, or 5 μM RA L-PGDS compared to mature fibrils. Data plotted are averages with SD (n=3). B. ThT fluorescence assay for inhibition of 50 μM (R124H) TGFBIp peptide grown for >65 h in the presence of 5 μM WT L-PGDS and 5 μM RA L-PGDS. C. Transmission electron micrographs of 50 μM mature R124C fibrils only (left), and R124C treated with 5 μM WT L-PGDS (middle), and 5 μM RA L-PGDS (right). Scale bar is 100 nm.

FIG. 7 shows near-complete inhibition of aggregation of 100 μM Tau peptide VK26 (circle), in the presence of 10 μM RA L-PGDS (square) and 5 μM RA L-PGDS (triangle) over a period of >68 h detected by ThT assay. Excitation wavelength: 450 nm, emission wavelength: 485 nm.

### DETAILED DESCRIPTION OF THE INVENTION

Further details of the invention will now be described with reference to the following non-limiting examples. Unless otherwise defined herein, technical and scientific terms used in the present description have the meanings that are commonly understood by those of ordinary skill in the art.

### A. Definitions

As used herein, the singular forms “a,” “an,” and “the” include plural referents unless the context clearly dictates otherwise.

As used herein, the term “comprising” may include the embodiments “consisting of” and “consisting essentially of”. The terms “comprise(s),” “include(s),” “having,” “has,” “can,” “contain(s),” and variants thereof, as used herein, are intended to be open-ended transitional phrases that require the presence of the named ingredients/steps and permit the presence of other ingredients/steps. However, such description should be construed as also describing compositions, mixtures, or processes as “consisting of” and “consisting essentially of” the enumerated ingredients/steps, which allows the presence of only the named ingredients/steps, along with any impurities that might result therefrom, and excludes other ingredients/steps.

As used herein, the term “subject” refers to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has or is diagnosed of having a particular disease, for example, non-tuberculous mycobacteria infection.

As used herein, the term “therapeutically effective amount” of a drug refers to an amount of the drug that, when administered to a subject with a specified disorder or disease, is sufficient to have the intended effect, e.g., treatment, alleviation, amelioration, palliation or elimination of one or more manifestations of the specified disorder or disease in the subject. A therapeutic effect does not necessarily occur by administration of one dose, and may occur only after administration of a series of doses. Thus, a therapeutically effective amount may be administered in one or more administrations. The therapeutically effective amount will depend in part on the nature of the drug, the manner and route of administration, the stage and severity of the disease being treated, the weight and general state of health of the subject, and the judgment of the prescribing physician.

As used herein, the term “treatment” refers to ameliorating, therapeutic or curative treatment.

As used herein, the term “amyloid-related disease” refers to a disease resulting from the aggregation of amyloid fibrils in tissue. Examples of amyloid-related diseases include but are not limited to corneal dystrophy (CD) such as TGFBI-related CD, Alzheimer's disease (AD), frontotemporal dementia (FTD), Down's syndrome (DS), Pick disease (PiD), argyrophilic grain disease (AGD), progressive supranuclear palsy (PSP), corticobasal degeneration (CBD), Huntington's disease (HD), Prion disease (PrD), cataracts, bacterial infections caused by fibril-producing bacteria, Type 2 diabetes and Parkinson's disease (PD). Examples of fibril-producing bacteria that are known in the art, including but not limited to phylum such as Proteobacteria (Alpha-, Beta-, Gamma- and Delta-proteobacteria), Bacteriodetes, Chloroflexi, Firmicutes and Actinobacteria. More particular examples of fibril-producing bacteria include Bacteroides fragilis, Chloroflexus aggregans, Bacillus licheniformis, Salmonella typhimurium, Enterobacter sakazaki, Aeromonas caviae, Xanthomonas anxidopidos, Chromobacterium violaceum, Burkholderia gladioli, Burkholderia pseudomallei, Ralstonia pikettii, Stenotrophomonas maltophilia, Escherichia coli (E. coli), Salmonella enterica, Pseudomonas aeruginosa, Pseudomonas fluorescens, Pseudomonas putida, Staphylococcus aureus, and Mycobacterium tuberculosis.

### B. RA L-PGDS

Lipocalin-type prostaglandin D synthase (L-PGDS) is a multifunctional protein abundant in the human cerebrospinal fluid. L-PGDS functions as a prostaglandin D₂ (PGD) synthase (producing PGD2, a prostaglandin involved in anti-tumorigenesis activity), a transporter for lipophilic ligands, and a pseudo-peroxidase that can bind to heme and function as a reactive oxygen species (ROS) scavenger, limiting the release of cytotoxic, free ROS in cellular environments (Lim et al., 2013; Phillips et al., 2020). L-PGDS is also reported to be a key amyloid β chaperone in the brain, inhibiting Amyloid β (Aβ) aggregation and functioning as disaggregase of Aβ fibrils (Kannaian et al., 2019). L-PGDS is not natively expressed in the cornea (Dyrlund et al., 2012) and thus administration of this multifunctional protein for CD intervention may introduce unwanted toxicity and side effects. The full length amino acid sequence of the wild type L-PGDS is set forth in SEQ ID NO: 12.

In the present invention, a truncated version of the wild type L-PGDS without the signal peptide is used (SEQ ID NO: 3). RAL-PGDS is a mutant of the truncated wild type L-PGDS comprising K58R and C65A double mutations, and its amino acid sequence is set forth in SEQ ID NO: 5. C65A L-PGDS, which comprises only a single C65A mutation is also used in the present invention, and its amino acid sequence is set forth in SEQ ID NO: 4. The numbering of the amino acid positions in the present application refers to the full length wild type L-PGDS having an amino acid sequence of SEQ ID NO: 12.

### C. Use of the RA L-PGDS Mutant

Due to its activity in disaggregating amyloids such as TGFBI fibrils, Aβ fibrils and bacteria amyloids, RA L-PGDS may be used to treat amyloid related diseases corneal dystrophy (CD) such as TGFBI-related CD, Alzheimer's disease (AD), frontotemporal dementia (FTD), Down's syndrome (DS), Pick disease (PiD), argyrophilic grain disease (AGD), progressive supranuclear palsy (PSP), corticobasal degeneration (CBD), Huntington's disease (HD), Prion disease (PrD), cataracts, bacterial infections caused by fibril-producing bacteria, Type 2 diabetes and Parkinson's disease (PD). In some embodiments, the fibril-producing bacteria is selected from a group consisting of Proteobacteria (Alpha-, Beta-, Gamma- and Delta-proteobacteria), Bacteriodetes, Chloroflexi, Firmicutes and Actinobacteria. In some embodiments the fibril-producing bacteria are selected from the group comprising Bacteroides fragilis, Chloroflexus aggregans, Bacillus licheniformis, Salmonella typhimurium, Enterobacter sakazaki, Aeromonas caviae, Xanthomonas anxidopidos, Chromobacterium violaceum, Burkholderia gladioli, Burkholderia pseudomallei, Ralstonia pikettii, Stenotrophomonas maltophilia, Escherichia coli (E. coli), Salmonella enterica, Pseudomonas aeruginosa, Pseudomonas fluorescens, Pseudomonas putida, Staphylococcus aureus and Mycobacterium tuberculosis.

For example, RA L-PGDS may be used as a dual therapeutic strategy to target both TGFBI-related CD and bacterial infections that can occur in the cornea. These two conditions can have overlapping symptoms and complications, such as weakened cornea due to corneal dystrophies making it more susceptible to bacterial infections. Moreover, some types of corneal dystrophies can cause corneal erosions, which can provide an entry point for bacteria. Therefore, a treatment that can address both conditions would be highly desirable.

The advantage of RA L-PGDS over other amyloid inhibitors is that it is a protein inhibitor, which means it can be designed to target specific amyloid proteins without affecting other microbial species or the host's microbiome. Moreover, protein amyloid inhibitors can be more stable and have better pharmacokinetics than small molecules like Epigallocatechin gallate (EGCG), which can have a short half-life and require high concentrations to be effective.

Further, as RA L-PGDS is able to disaggregate bacterial amyloids, it may also be used to prevent the growth of harmful fibril-producing bacteria. For example, it may be used to prevent the growth of harmful fibril-producing bacteria present on medical devices such as catheters and implants by inhibiting the formation of biofilm on these devices, which could potentially reduce the incidence of infections associated with catheter use in hospitals. In another embodiment, it may be used to prevent the growth of harmful fibril-producing bacteria present in environmental settings such as oil spills by preventing or disrupting the formation of biofilms, thereby reducing their impact on the environment. The presence of oil can lead to the formation of microbial biofilms, which can exacerbate the damage caused by oil spills. The application of RA L-PGDS directly to the affected areas could target the formation and growth of these biofilms. In another embodiment, it may be used to prevent the growth of harmful fibril-producing bacteria present in food products, beverage products and personal care products. In another embodiments, RA L-PGDS may be used in combination with other antimicrobial agents to enhance their effectiveness, especially in disrupting biofilms that might inhibit the activity of other agents, forming a synergistic effect and allowing them to more effectively degrade the bacteria found within the biofilm.

It should be understood that any and all embodiments of the present disclosure can be combined with technical features in any other embodiment or multiple other embodiments to obtain additional embodiments under the premise of no conflict. The invention includes such combinations resulting in further embodiments.

### EXAMPLES

The examples and exemplary embodiments below are intended to be purely exemplary of the invention and should therefore not be considered to limit the invention in any way.

### Example 1. Materials and Methods

### Synthetic Peptides

Synthetic peptides of the 4th FAS-1 domain of TGFBIp (SEQ ID NO: 1), as well as its G623R mutant (SEQ ID NO: 2), were purchased from Synpeptide Co Ltd, Shanghai, China. Other variants of the TGFBIp peptides (R124C, R124H, 515-533, 591-614, 571-588), Tau peptide and Aβ (1-40) peptides were purchased from Biobasic Asia Pacific Pte Ltd, Singapore. Thioflavin T (ThT) was purchased from Sigma-Aldrich (Sigma-Aldrich Inc., MO).

### In-Vitro Uniform Amyloid Fibril Formation

The 23-amino acid long peptide TGFBIp G623R from the 4th FAS1 domain of TGFBIp with the substitution G623R (SEQ ID NO: 2) that is known to rapidly aggregate to form amyloid fibrils was used in this study. The peptide powder was dissolved (0.15 mg/ml) in PBS and allowed to form amyloid fibrils in a shaking incubator at 37° C. and 180 rpm with and without the addition of L-PGDS variants at different concentrations.

### Overexpression and Purification of Human WT L-PGDS and its Mutants

A glycerol stock of Rosetta 2 DE3, E. coli cells (Novagen) was transformed with pNIC-CH vectors containing the gene encoding truncated human wild-type L-PGDS (SEQ ID NO: 3) and a C-terminal hexa-histidine-tag. The transformed cells were grown at 37° C. in Luria-Bertani (LB) liquid media containing antibodies kanamycin and chloramphenicol in 1:1000 v/v ratio and induced with 0.5 mM IPTG at O.D₆₀₀ of 0.8. The crude L-PGDS was injected into the AKTA purifier Fast Performance Liquid Chromatography (FPLC) (GE Healthcare, USA), and further purified using Superdex 75 column in Phosphate buffered saline (PBS) buffer. Sodium dodecyl sulfate-polyacrylamide gel electrophoresis (SDS-PAGE) was run to check the purity of the different fractions obtained after the FPLC run.

A C65A single mutant (SEQ ID NO: 4) and a K58R+C65A double mutant (RA L-PGDS, SEQ ID NO: 5) of L-PGDS were constructed by site-directed mutagenesis. Plasmid of the C65A L-PGDS and RA L-PGDS were purchased from Biobasic Asia Pacific Pte Ltd. Both mutants were expressed and purified using the same protocol as WT L-PGDS but with ampicillin as the selection agent for RA L-PGDS plasmid-transformed cells.

Transmission Electron Microscopy to assess structural changes of amyloid fibrils Morphological analysis of the preformed G623R amyloid fibrils before and after treatment with L-PGDS or its mutants for 72 hours was investigated by Transmission electron microscopy (TEM) with a FEI T12 transmission electron microscope equipped with a 4K CCD camera (FEI) at a magnification of 49000× and electron dose of 20-30 e⁻/Å². 4 μl of amyloid fibril samples with and without L-PGDS treatment were applied onto the glow-discharged 400-mesh-size carbon grids for 1 minute and stained with 2% (v/v) uranyl acetate for 30 s before viewing under the microscope.

### Analysis of the EM Images

The end-to-end distance (D), contour length (L) and angles of the fibrils were measured using the ImageJ software 43. The persistence length (P) of the fibrils was obtained using the MS end-to-end distance processing tool in FiberApp software. The plot of end-to-end distance is fitted using the following equation:

$$( {D}^{2} ) = 4 PL [ 1 - \frac{2 P}{L} ( 1 - {e}^{( - \frac{L}{2 P} )} ) ]$$

### Thioflavin T (ThT) Assay

Wildtype TGFBIp peptide fibrillation and inhibition and disaggregation activity of all LPGDS variants were studied using ThT assays. ThT powder (Sigma Aldrich, Singapore) was dissolved in Mili-Q water and filtered using a 0.1 μm syringe filter to obtain a stock concentration of 2.3 mM. The stock solution was further diluted with PBS to a working concentration of 200 μM. Fluorescence intensity for all ThT experiments were measured with λ_{excitation}=430 nm and λₑₘᵢₛₛᵢₒₙ=480 nm.

For inhibitory studies, fibril peptide (such as G623R and other TGFBIp variants, Aβ (1-40)) powder was freshly dissolved in filtered PBS to a stock concentration of 250 μM and reactions with final concentrations of 50 μM fibril peptide, 20 μM ThT with or without 10 μM L-PGDS (WT or mutants) treatments were prepared. Measurements were taken at 0 h and 48 h, at 37° C., to monitor the formation of the fibrils and the reactions were subjected to continuous shaking throughout the 37° C. incubation period.

For disaggregation assays, fibrils were grown by dissolving fibril peptide powder in filtered PBS to a concentration of 250 μM and incubated at 37° C., with continuous shaking, for 72 hours. Disaggregase reactions were prepared in a similar manner to inhibitory studies and measurements were taken at 0 h and 48 h at 37° C. to monitor the disaggregation activity of L-PGDS variants.

The percentage of ThT fluorescence intensity was calculated using the following formula:

$$\% ThT = \frac{{F}_{G + L}}{{F}_{G}} \times 100 \%$$

where % ThT is the percentage of ThT fluorescence intensity, F_{G+L} is the average ThT fluorescence intensity of L-PGDS treated reactions and F_{G} is the average ThT fluorescence intensity of the control reactions.

### Heme Binding and Peroxidase Assay

2.5 mM heme stock was prepared in 0.1% NaOH and was further diluted with 0.1% NaOH into 250 μM working concentration. Heme was added at 1:1 molar ratio to 5 μM L-PGDS in 50 μM sodium phosphate buffer and incubated at room temperature for 2 hours. Absorbances of the triplicate reactions were measured in the range 350-650 nm with an increment of 5 nm on the microplate reader, at 25° C.

Peroxidase activity of the L-PGDS/heme complexes were determined by measuring the oxidation of 3,3 0,5,50-tetramethylbenzidine (TMB) High Sensitivity Substrate Solution (BioLegend Inc., USA) at 652 nm. TMB was added to the triplicate LPGDS/heme reactions from the heme binding assay and peroxidase activity was monitored for 1 hour on the microplate reader, at 25° C., with 3 s orbital shaking before each measurement.

### Example 2. Inhibition and Disaggregation Activity of Mutant L-PGDS Against Mutant TGFBIp Fibrils

L-PGDS is not natively expressed in the central cornea and thus considerations as a TGFBIp-associated Lattice corneal dystrophies (LCD) treatment imposes toxicity concerns. Two general strategies were employed to minimize L-PGDS toxicity in the cornea by either directly reducing off-target interactions of the multifunctional protein or indirectly by improving the efficacy of L-PGDS and, hence lowering the effective therapeutic dose.

Cys 65 of L-PGDS is the catalytic residue for the prostaglandin D₂ (PGD) synthase function and C65A substitution has been demonstrated to inactive the synthase functionality. Additionally, release of PGD2 and likely other lipophilic ligands requires plasma membrane interactions which can contribute to toxicity effects (Lim et al., 2013). The C65A mutation appears to have no effect on the inhibition and disaggregation activity in Aβ. Thus, C65A L-PGDS has the potential to be a chaperone for amyloidogenic TGFBIp with reduced off-target events.

Extended conformation and higher pKa of the basic entities in arginine side chains often allows for increased and stable ionic interaction in comparison to lysine (Sokalingam et al., 2012). In the context of PGD synthase function, a K59A mutation has been observed to increase enzymatic activity by two-fold as the mutation mimics the electrostatic induced translocation of K59 out of the catalytic pocket for product capture (Zhou et al., 2010). Mutations of K59 to arginine is thus considered a sensitive endeavour as it could have the capability to better accommodate lipophilic ligands and resultant membrane interactions even in synthase inactivated C65A-L-PGDS. With these considerations, a single C65A and a double K58R and C65A mutant (RA) of L-PGDS were pursued to be synthesized and investigated for improved LPGDS disaggregase and inhibitory activity against G623R, a hereditary mutant isoform clinically elucidated in 2009 (Gruenauer-Kloevekorn et al., 2009).

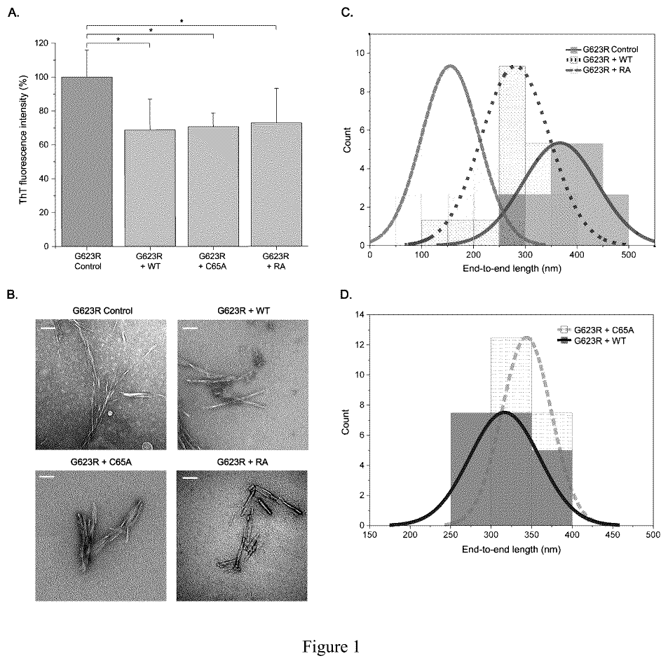

ThT fluorescence measurements revealed that both C65A L-PGDS and RA L-PGDS disaggregated G623R fibrils at 1:5 ratio, matching the activity of WT-L-PGDS, as observed by significant (p<0.05) reductions in relative fluorescence after 48 h (FIG. 1A). No significant differences in fluorescence were observed within the mutants, with WT, C65A and RAL-PGDS, reducing ThT fluorescence to 68.78%±18.20% (SD), 70.56%±8.18% (SD) and 72.78%±20.42% (SD) respectively.

TEM micrographs (FIG. 1B) of control G623R fibrils displayed long, largely bundled fibrils with multiple twists, indicated by white contrasts within the fibrils. Treatment with all L-PGDS mutants appeared to reduce the length and density of the fibrils with RA L-PGDS treatment producing seemingly shorter fibrils as opposed to WT L-PGDS. Further analysis of TEM micrographs by measuring lengths of control and RA L-PGDS or WT L-PGDS treated fibrils showed that RA-L-PGDS treatment did indeed produce shorter fibrils as opposed to WT L-PGDS (FIG. 1C). Distribution of fibril lengths in C65A L-PGDS or WT L-PGDS treated fibrils demonstrated minimal shifts, with WT L-PGDS producing marginally shorter fibrils (FIG. 1D). Thus, micrograph analyses were able to uncover the improved disaggregation function of RA L-PGDS not captured in ThT assays.

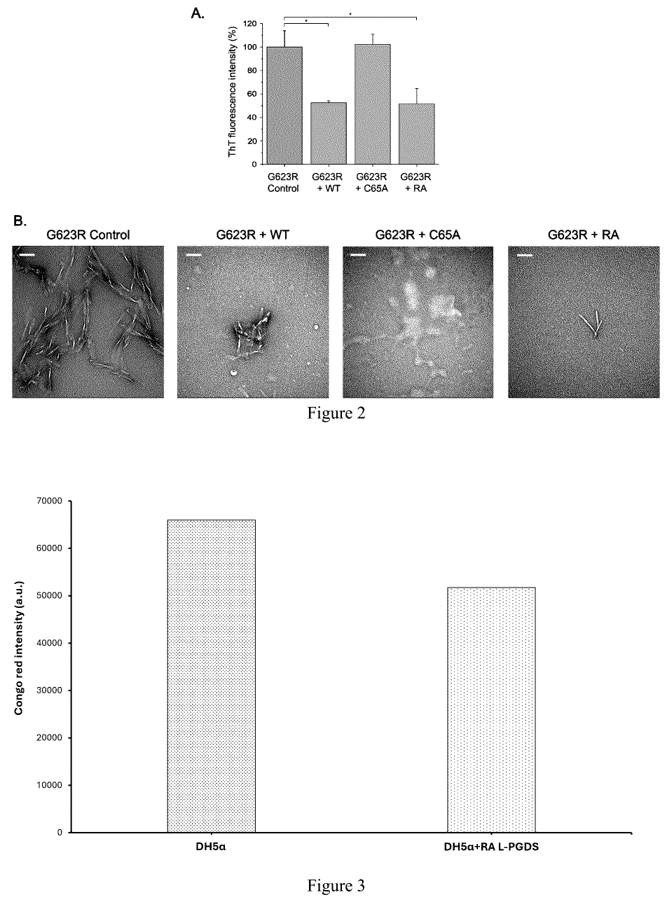

Inhibition assays of G623R fibrillation by L-PGDS mutants (FIG. 2A) showed that only RA L-PGDS retained G623R fibrillation inhibition activity observed in WT-LPGDS. WT and RA L-PGDS treatment significantly lowered (p<0.05) ThT fluorescence intensity to 52.52%±1.66% (SD) and 51.44%±13.20% (SD) respectively, relative to the G623R control after 48 h. C65A L-PGDS appeared to have no effect on G623R fibrillation and exhibited a 102.39%±8.58% (SD) fluorescence intensity relative to the control.

Inhibitory activity of WT and RA L-PGDS were visually confirmed by the TEM micrographs (FIG. 2B) highlighting reduced fibril density and length. TEM micrographs of C65A L-PGDS treatment revealed the presence of low contrast gel-like aggregates, dissimilar to the high contrast amorphous aggregates observed in WT L-PGDS disaggregation images (FIG. 1B), with the absence of fibrils.

Taken together, RA L-PGDS showed improved disaggregation activity of G623R compared with WT and C65A L-PGDS, and also better inhibition of G623R fibrillation compared with C65A L-PGDS.

### Example 3. Bacterial Amyloid Disaggregation by RA L-PGDS

Further research on the potential of RA L-PGDS to disaggregate bacterial amyloid was conducted. 50 μM RA L-PGDS was incubated with DH5a cells and the effects observed on amyloid formation. After incubation, the bacteria cells were grown on a Congo red stain agar plate and illuminated with near infrared light to detect the formation of bacterial amyloid. Specifically, the congo red intensity was measured using a spectrophotometer by measuring its absorbance at around 488 nm. Interestingly, it was found that the addition of RA L-PGDS in DH5α cells resulted in a ˜20% decrease in Congo red intensity, as demonstrated in FIG. 3. This suggests that RA L-PGDS has the ability to disaggregate the bacteria amyloid produced by the DH5α cells, as Congo red dye is a known amyloid-specific dye.

### Example 4. Inhibition and Disaggregation Activity of L-PGDS Mutant Against Aβ (1-40) Fibrils

ThT disaggregation assay and inhibitory assay as described in Example 1 were performed with Aβ (1-40) (SEQ ID NO: 13) to test the function of L-PGDS mutants.

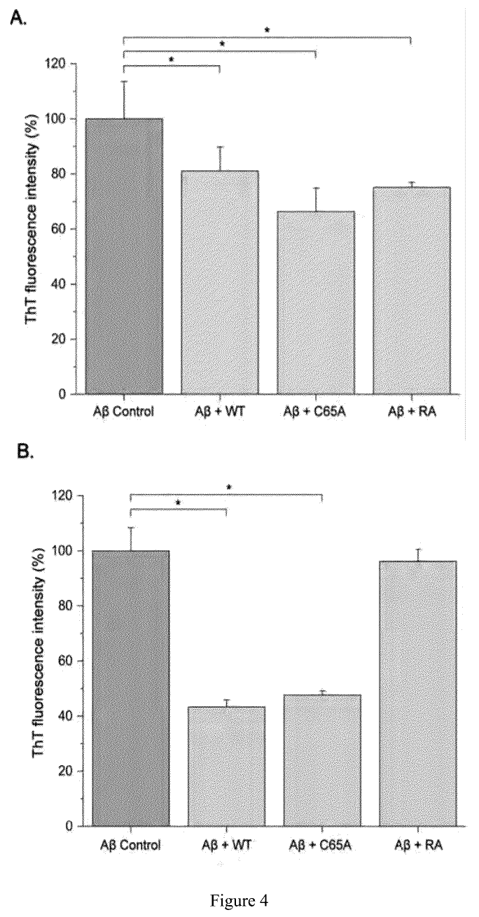

ThT disaggregation assay (FIG. 4A) showed significant (p<0.05) retention of disaggregation activity across all L-PGDS mutants after 24 h of incubation, as observed with G623R fibrils. Inhibitory ThT assay (FIG. 4B) revealed that C65A and WT L-PGDS significantly (p<0.05) inhibited Aβ (1-40), with no significant differences between them. These observations verified that C65A L-PGDS was indeed functional. However, RA-L-PGDS is unable to inhibit the fibrillation of Aβ (1-40), as seen from the 96.13%±4.40% (SD) fluorescence relative to Aβ (1-40) control.

### Example 5. Heme Binding and Peroxidase Activity of L-PGDS Mutants

Peroxidase activity of L-PGDS-heme complexes is regarded as a protective feature due to subsequent ROS scavenging in the L-PGDS pocket by cystines or ligands bound to the protein, reducing toxic peroxide levels. Lys 58 and Cys 65 are expected to be involved in accessory heme interactions and ROS scavenging respectively, with M145 being the key residue for heme binding (Phillips et al., 2020). The retention of heme binding and peroxidase activity in the two L-PGDS mutants, lacking the mentioned accessory residues, was inspected to examine the preservation of cell protective scavenger activity.

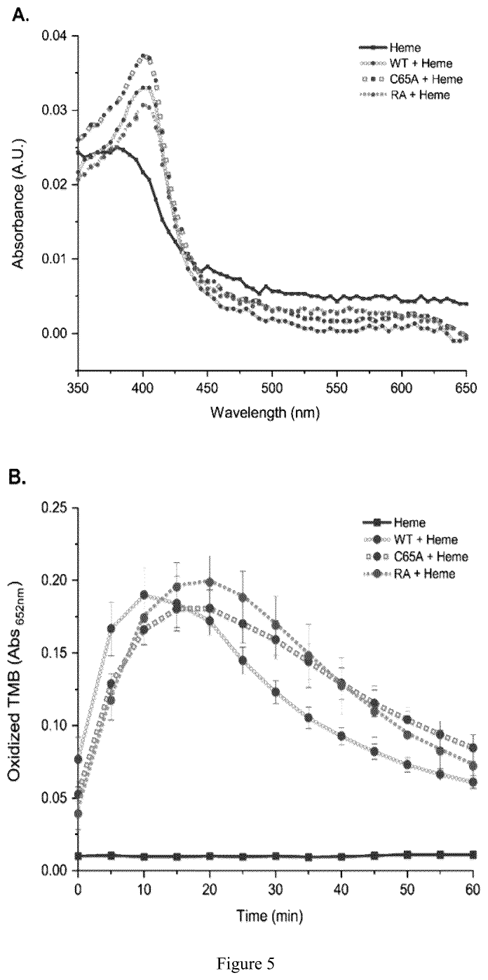

Absorbance scanning in the UV/Vis spectrum showed that C65A and RA L-PGDS maintained the heme binding observed in WT L-PGDS as shown by the red shift of the Soret band from 390 nm to 405 nm upon addition of L-PGDS mutants to free heme (FIG. 5A). TMB oxidation monitoring for 1 h highlighted the retention of peroxidase activity in both mutants with no significant changes in peak activities (FIG. 5B). Thus, these observations collectively informed on the retention of heme binding and peroxidase activity in both L-PGDS mutants.

### Example 6. Inhibition Effect of WT L-PGDS and RA L-PGDS on More Mutated Peptides Associated with TGFBIp-Induced Corneal Dystrophy

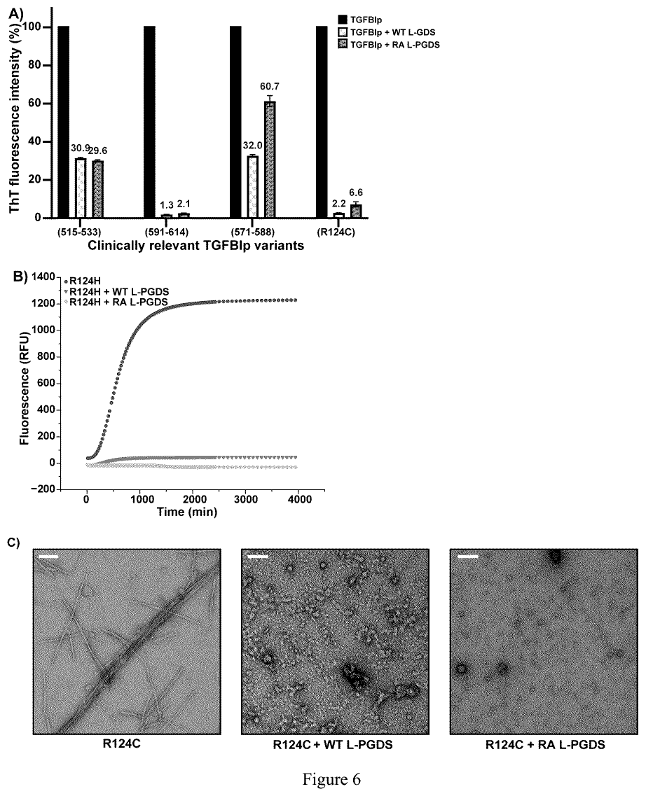

Effects of WT L-PGDS and RA L-PGDS on other clinically relevant TGFBIp variants 515-533 (SEQ ID NO: 6), 591-614 (SEQ ID NO: 7), 571-588 (SEQ ID NO: 8) and R124C (SEQ ID NO: 9) were also compared using the ThT inhibitory assay described in Example 1. It was observed that both WT L-PGDS and RA L-PGDS showed inhibition of the tested TGFBIp variants, indicating potential therapeutic impact of both mutants (FIG. 6A). The continuous inhibitory effects of WT L-PGDS and RA L-PGDS on the TGFBIp variant R124H (SEQ ID NO: 10) was further shown in FIG. 6B, wherein RA L-PGDS exhibited slightly better inhibition effects compared with WT-LPGDS. The disaggregation results of R124C peptide was further confirmed with TEM imaging (FIG. 6C).

### Example 7. Inhibition Effect of WT L-PGDS and RA L-PGDS on Tau Protein Aggregation

WT L-PGDS and RA L-PGDS were tested on Tau peptide VK26 (SEQ ID NO: 11). Briefly, Tau VK26 protein powder was freshly dissolved in filtered PBS to a stock concentration of 250 μM and reactions with final concentrations of 100 μM Tau peptide, 20 μM ThT without RA L-PGDS treatment, or with 5 μM or 10 μM RA L-PGDS treatments were prepared. Measurements were taken every 10 minutes up to 40 hours, at 37° C., to monitor the formation of the fibrils and the reactions were subjected to continuous shaking throughout the 37° C. incubation period. Fluorescence intensity was measured with λ_{excitation}=450 nm and λₑₘᵢₛₛᵢₒₙ=485 nm.

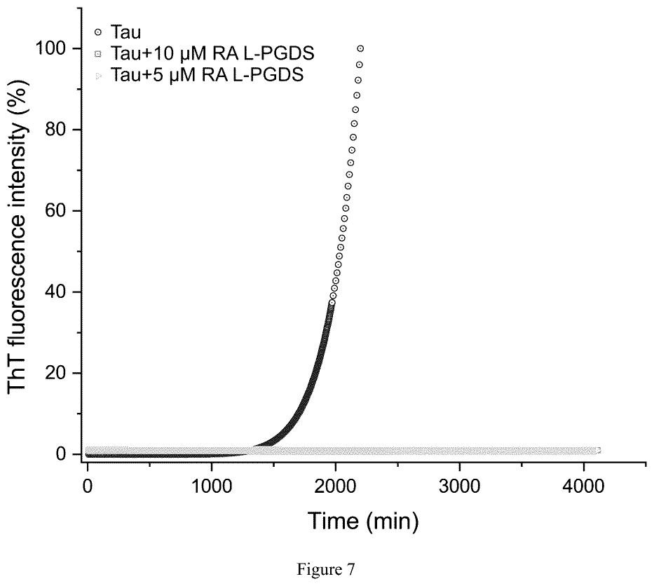

As shown in FIG. 7, RA L-PGDS almost completely inhibited the aggregation of the Tau protein.

For one skilled in the art, various modifications and changes may be made to the present disclosure. Those skilled in the art should understand that any amendments, equivalent replacements, improvements, and so on, made within the spirit and principle of the present disclosure, should be covered within the scope of protection of the present disclosure.

### BIBLIOGRAPHY

1. Chao-Shern C, DeDionisio L A, Jang J H, Chan C C, Thompson V, Christie K, Nesbit M A, McMullen C B T. Evaluation of TGFBI corneal dystrophy and molecular diagnostic testing. Eye (Lond). 2019 June; 33 (6): 874-881. doi: 10.1038/s41433-019-0346-x.
2. Lakshminarayanan R, Chaurasia S S, Anandalakshmi V, Chai S M, Murugan E, Vithana E N, Beuerman R W, Mehta J S. Clinical and genetic aspects of the TGFBI-associated corneal dystrophies. Ocul Surf. 2014 October; 12 (4): 234-51. doi: 10.1016/j.jtos.2013.12.002.
3. Nielsen N S, Poulsen E T, Lukassen M V, Chao Shern C, Mogensen E H, Weberskov C E, DeDionisio L, Schauser L, Moore T C B, Otzen D E, Hjortdal J, Enghild J J. Biochemical mechanisms of aggregation in TGFBI-linked corneal dystrophies. Prog Retin Eye Res. 2020 July; 77:100843. doi: 10.1016/j.preteyeres.2020.100843.
4. Han K E, Choi S I, Kim T I, Maeng Y S, Stulting R D, Ji Y W, Kim E K. Pathogenesis and treatments of TGFBI corneal dystrophies. Prog Retin Eye Res. 2016 January; 50:67-88. doi: 10.1016/j.preteyeres.2015.11.002.
5. Kheir V, Cortés-González V, Zenteno J C, Schorderet D F. Mutation update: TGFBI pathogenic and likely pathogenic variants in corneal dystrophies. Hum Mutat. 2019 June; 40 (6): 675-693. doi: 10.1002/humu.23737.
6. Stone E M, Mathers W D, Rosenwasser G O, Holland E J, Folberg R, Krachmer J H, Nichols B E, Gorevic P D, Taylor C M, Streb L M, et al. Three autosomal dominant corneal dystrophies map to chromosome 5q. Nat Genet. 1994 January; 6 (1): 47-51. doi: 10.1038/ng0194-47.
7. Munier F L, Korvatska E, Djemaï A, Le Paslier D, Zografos L, Pescia G, Schorderet D F. Kerato-epithelin mutations in four 5q31-linked corneal dystrophies. Nat Genet. 1997 March; 15 (3): 247-51. doi: 10.1038/ng0397-247.
8. Kannabiran C, Sridhar M S, Chakravarthi S K, Vemuganti G K, Lakshmipathi M. Genotype-phenotype correlation in 2 Indian families with severe granular corneal dystrophy. Arch Ophthalmol. 2005 August; 123 (8): 1127-33. doi: 10.1001/archopht.123.8.1127.
9. Venkatraman A, Dutta B, Murugan E, Piliang H, Lakshminaryanan R, Sook Yee A C, Pervushin K V, Sze S K, Mehta J S. Proteomic Analysis of Amyloid Corneal Aggregates from TGFBI-H626R Lattice Corneal Dystrophy Patient Implicates Serine-Protease HTRA1 in Mutation-Specific Pathogenesis of TGFBIp. J Proteome Res. 2017 Aug. 4; 16 (8): 2899-2913. doi: 10.1021/acs.jproteome.7b00188.
10. Anandalakshmi V, Murugan E, Leng E G T, Ting L W, Chaurasia S S, Yamazaki T, Nagashima T, George B L, Peh G S L, Pervushin K, Lakshminarayanan R, Mehta J S. Effect of position-specific single-point mutations and biophysical characterization of amyloidogenic peptide fragments identified from lattice corneal dystrophy patients. Biochem J. 2017 May 9; 474 (10): 1705-1725. doi: 10.1042/BCJ20170125.
11. Mannis M J, Krachmer J H, Rodrigues M M, Pardos G J. Polymorphic amyloid degeneration of the cornea. A clinical and histopathologic study. Arch Ophthalmol. 1981 July; 99 (7): 1217-23. doi: 10.1001/archopht.1981.03930020091008.
12. Molia L M, Lanier J D, Font R L. Posterior polymorphous dystrophy associated with posterior amyloid degeneration of the cornea. Am J Ophthalmol. 1999 January; 127 (1): 86-8. doi: 10.1016/s0002-9394 (98) 00276-1.
13. Yu J T, Tan L, Hardy J. Apolipoprotein E in Alzheimer's disease: an update. Annu Rev Neurosci. 2014; 37:79-100. doi: 10.1146/annurev-neuro-071013-014300.
14. Kim J, Basak J M, Holtzman D M. The role of apolipoprotein E in Alzheimer's disease. Neuron. 2009 Aug. 13; 63 (3): 287-303. doi: 10.1016/j.neuron.2009.06.026.
15. Underwood, E. The polluted Brain. Science Magazine 355, 342-345 (2017).
16. Dominy S S, Lynch C, Ermini F, Benedyk M, Marczyk A, Konradi A, Nguyen M, Haditsch U, Raha D, Griffin C, Holsinger L J, Arastu-Kapur S, Kaba S, Lee A, Ryder M I, Potempa B, Mydel P, Hellvard A, Adamowicz K, Hasturk H, Walker G D, Reynolds E C, Faull R L M, Curtis M A, Dragunow M, Potempa J. Porphyromonas gingivalis in Alzheimer's disease brains: Evidence for disease causation and treatment with small-molecule inhibitors. Sci Adv. 2019 Jan. 23; 5 (1): eaau3333. doi: 10.1126/sciadv.aau3333.
17. Lee J H, Chung S H, Stulting R D, Kim W C, Lee H K, Kim E K. Effects of corneal neovascularization on the manifestations of Avellino corneal dystrophy (granular corneal dystrophy type II). Cornea. 2006 September; 25 (8): 914-8. doi: 10.1097/01.ico.0000224645.89342.55.
18. Poulsen E T, Nielsen N S, Scavenius C, Mogensen E H, Risør M W, Runager K, Lukassen M V, Rasmussen C B, Christiansen G, Richner M, Vorum H, Enghild J J. The serine protease HtrA1 cleaves misfolded transforming growth factor β-induced protein (TGFBIp) and induces amyloid formation. J Biol Chem. 2019 Aug. 2; 294 (31): 11817-11828. doi: 10.1074/jbc.RA119.009050.
19. Wilson M R, Satapathy S, Jeong S, Fini M E. Clusterin, other extracellular chaperones, and eye disease. Prog Retin Eye Res. 2022 July; 89:101032. doi: 10.1016/j.preteyeres.2021.101032.
20. Peinado J R, Chaplot K, Jarvela T S, Barbieri E M, Shorter J, Lindberg I. Sequestration of TDP-43216-414 Aggregates by Cytoplasmic Expression of the proSAAS Chaperone. ACS Chem Neurosci. 2022 Jun. 1; 13 (11): 1651-1665. doi: 10.1021/acschemneuro.2c00156.
21. Kannaian B, Sharma B, Phillips M, Chowdhury A, Manimekalai M S S, Adav S S, Ng J T Y, Kumar A, Lim S, Mu Y, Sze S K, Grüber G, Pervushin K. Abundant neuroprotective chaperone Lipocalin-type prostaglandin D synthase (L-PGDS) disassembles the Amyloid-β fibrils. Sci Rep. 2019 Aug. 29; 9 (1): 12579. doi: 10.1038/s41598-019-48819-5.
22. Meyer-Luehmann M, Stalder M, Herzig M C, Kaeser S A, Kohler E, Pfeifer M, Boncristiano S, Mathews P M, Mercken M, Abramowski D, Staufenbiel M, Jucker M. Extracellular amyloid formation and associated pathology in neural grafts. Nat Neurosci. 2003 April; 6 (4): 370-7. doi: 10.1038/nn1022.
23. Low K J Y, Venkatraman A, Mehta J S, Pervushin K. Molecular mechanisms of amyloid disaggregation. J Adv Res. 2021 May 20; 36:113-132. doi: 10.1016/j.jare.2021.05.007.
24. Gates S N, Yokom A L, Lin J, Jackrel M E, Rizo A N, Kendsersky N M, Buell C E, Sweeny E A, Mack K L, Chuang E, Torrente M P, Su M, Shorter J, Southworth D R. Ratchet-like polypeptide translocation mechanism of the AAA+ disaggregase Hsp104. Science. 2017 Jul. 21; 357 (6348): 273-279. doi: 10.1126/science.aan1052.
25. De Los Rios P, Ben-Zvi A, Slutsky O, Azem A, Goloubinoff P. Hsp70 chaperones accelerate protein translocation and the unfolding of stable protein aggregates by entropic pulling. Proc Natl Acad Sci USA. 2006 Apr. 18; 103 (16): 6166-71. doi: 10.1073/pnas.0510496103.
26. Kanekiyo T, Ban T, Aritake K, Huang Z L, Qu W M, Okazaki I, Mohri I, Murayama S, Ozono K, Taniike M, Goto Y, Urade Y. Lipocalin-type prostaglandin D synthase/beta-trace is a major amyloid beta-chaperone in human cerebrospinal fluid. Proc Natl Acad Sci USA. 2007 Apr. 10; 104 (15): 6412-7. doi: 10.1073/pnas.0701585104.
27. Reiber H. Dynamics of brain-derived proteins in cerebrospinal fluid. Clin Chim Acta. 2001 Aug. 20; 310 (2): 173-86. doi: 10.1016/s0009-8981 (01) 00573-3.
28. Dyrlund T F, Poulsen E T, Scavenius C, Nikolajsen C L, Thøgersen I B, Vorum H, Enghild J J. Human cornea proteome: identification and quantitation of the proteins of the three main layers including epithelium, stroma, and endothelium. J Proteome Res. 2012 Aug. 3; 11 (8): 4231-9. doi: 10.1021/pr300358k.
29. Klintworth, G. K. (2009). Corneal dystrophies. Orphanet Journal of Rare Diseases, 4 (1). doi: 10.1186/1750-1172-4-7.
30. Lim S M, Chen D, Teo H, Roos A, Jansson A E, Nyman T, Trésaugues L, Pervushin K, Nordlund P. Structural and dynamic insights into substrate binding and catalysis of human lipocalin prostaglandin D synthase. J Lipid Res. 2013 June; 54 (6): 1630-1643. doi: 10.1194/jlr.M035410.
31. Phillips M, Kannaian B, Yang J N T, Kather R, Yuguang M, Harmer J R, Pervushin K. Amyloid β chaperone-lipocalin-type prostaglandin D synthase acts as a peroxidase in the presence of heme. Biochem J. 2020 Apr. 17; 477 (7): 1227-1240. doi: 10.1042/BCJ20190536.
32. Sokalingam S, Raghunathan G, Soundrarajan N, Lee S G. A study on the effect of surface lysine to arginine mutagenesis on protein stability and structure using green fluorescent protein. PLOS One. 2012; 7 (7): e40410. doi: 10.1371/journal.pone.0040410.
33. Zhou Y, Shaw N, Li Y, Zhao Y, Zhang R, Liu Z J. Structure-function analysis of human I-prostaglandin D synthase bound with fatty acid molecules. FASEB J. 2010 December; 24 (12): 4668-77. doi: 10.1096/fj. 10-164863.

1. An isolated Lipocalin-type prostaglandin D synthase (L-PGDS) polypeptide mutant consisting of the amino acid sequence set forth in SEQ ID NO: 5.

2. A composition comprising the isolated L-PGDS polypeptide mutant of claim 1 and at least one pharmaceutically acceptable excipient, diluent or carrier.

3. An in vitro method of inhibiting the growth of a fibril-producing bacteria, comprising contacting an effective amount of the L-PGDS polypeptide mutant of claim 1 with the bacteria.

4. The method of claim 3, wherein the bacteria are present in medical devices, environment settings such as soil and oil spill, food products, beverage products and personal care products.

5. The method of claim 3, wherein the fibril-producing bacteria is selected from a group consisting of Proteobacteria (Alpha-, Beta-, Gamma- and Delta-proteobacteria), Bacteriodetes, Chloroflexi, Firmicutes and Actinobacteria.

6. The method of claim 5, wherein the fibril-producing bacteria is selected from a group consisting of Bacteroides fragilis, Chloroflexus aggregans, Bacillus licheniformis, Salmonella typhimurium, Enterobacter sakazaki, Aeromonas caviae, Xanthomonas anxidopidos, Chromobacterium violaceum, Burkholderia gladioli, Burkholderia pseudomallei, Ralstonia pikettii, Stenotrophomonas maltophilia, Escherichia coli (E. coli), Salmonella enterica, Pseudomonas aeruginosa, Pseudomonas fluorescens, Pseudomonas putida, Staphylococcus aureus and Mycobacterium tuberculosis.

7. A method of treating an amyloid-related disease in a subject, comprising administering a therapeutically effective amount of the L-PGDS polypeptide mutant of claim 1 to the subject.

8. The method of claim 7, wherein the amyloid-related disease is selected from the group comprising corneal dystrophy (CD), Alzheimer's disease (AD), frontotemporal dementia (FTD), Down's syndrome (DS), Pick disease (PiD), argyrophilic grain disease (AGD), progressive supranuclear palsy (PSP), corticobasal degeneration (CBD), Huntington's disease (HD), Prion disease (PrD), cataracts, bacterial infections caused by fibril-producing bacteria, Type 2 diabetes and Parkinson's disease (PD).

9. The method of claim 8, wherein the CD is Transforming growth beta induced (TGFBI)-related CD, or the fibril-producing bacteria is selected from the group consisting of Proteobacteria (Alpha-, Beta-, Gamma- and Delta-proteobacteria), Bacteriodetes, Chloroflexi, Firmicutes and Actinobacteria.

10. The method of claim 9, wherein the fibril-producing bacteria is selected from the group consisting of Bacteroides fragilis, Chloroflexus aggregans, Bacillus licheniformis, Salmonella typhimurium, Enterobacter sakazaki, Aeromonas caviae, Xanthomonas anxidopidos, Chromobacterium violaceum, Burkholderia gladioli, Burkholderia pseudomallei, Ralstonia pikettii, Stenotrophomonas maltophilia, Escherichia coli (E. coli), Salmonella enterica, Pseudomonas aeruginosa, Pseudomonas fluorescens, Pseudomonas putida, Staphylococcus aureus, and Mycobacterium tuberculosis.

11. A method of treating an amyloid-related disease in a subject, comprising administering a therapeutically effective amount of the composition of claim 2 to the subject.

12. The method of claim 11, wherein the amyloid-related disease is selected from the group comprising corneal dystrophy (CD), Alzheimer's disease (AD), frontotemporal dementia (FTD), Down's syndrome (DS), Pick disease (PiD), argyrophilic grain disease (AGD), progressive supranuclear palsy (PSP), corticobasal degeneration (CBD), Huntington's disease (HD), Prion disease (PrD), cataracts, bacterial infections caused by fibril-producing bacteria, Type 2 diabetes and Parkinson's disease (PD).

13. The method of claim 12, wherein the CD is Transforming growth beta induced (TGFBI)-related CD, or the fibril-producing bacteria is selected from the group consisting of Proteobacteria (Alpha-, Beta-, Gamma- and Delta-proteobacteria), Bacteriodetes, Chloroflexi, Firmicutes and Actinobacteria.

14. The method of claim 13, wherein the fibril-producing bacteria is selected from the group consisting of Bacteroides fragilis, Chloroflexus aggregans, Bacillus licheniformis, Salmonella typhimurium, Enterobacter sakazaki, Aeromonas caviae, Xanthomonas anxidopidos, Chromobacterium violaceum, Burkholderia gladioli, Burkholderia pseudomallei, Ralstonia pikettii, Stenotrophomonas maltophilia, Escherichia coli (E. coli), Salmonella enterica, Pseudomonas aeruginosa, Pseudomonas fluorescens, Pseudomonas putida, Staphylococcus aureus, and Mycobacterium tuberculosis.

15. An in vitro method of inhibiting the growth of a fibril-producing bacteria, comprising contacting an effective amount of the composition of claim 2 with the bacteria.

16. The method of claim 15, wherein the bacteria are present in medical devices, environment settings such as soil and oil spill, food products, beverage products and personal care products.

17. The method of claim 15, wherein the fibril-producing bacteria is selected from a group consisting of Proteobacteria (Alpha-, Beta-, Gamma- and Delta-proteobacteria), Bacteriodetes, Chloroflexi, Firmicutes and Actinobacteria.

18. The method of claim 17, wherein the fibril-producing bacteria is selected from a group consisting of Bacteroides fragilis, Chloroflexus aggregans, Bacillus licheniformis, Salmonella typhimurium, Enterobacter sakazaki, Aeromonas caviae, Xanthomonas anxidopidos, Chromobacterium violaceum, Burkholderia gladioli, Burkholderia pseudomallei, Ralstonia pikettii, Stenotrophomonas maltophilia, Escherichia coli (E. coli), Salmonella enterica, Pseudomonas aeruginosa, Pseudomonas fluorescens, Pseudomonas putida, Staphylococcus aureus and Mycobacterium tuberculosis.
